(19) United States
(12) Patent Application Publication
(10) Pub. No.: US 2022/0195172 A1
(43) Pub. Date: Jun. 23, 2022

(54) HYDROPHILIC POLYMER COATINGS WITH DURABLE LUBRICITY AND COMPOSITIONS AND METHODS THEREOF

(71) Applicant: HydroGlyde Coatings LLC, Brookline, MA (US)

(72) Inventors: Stacy Lee Chin, Brookline, MA (US); Benjamin Goldman Cooper, Lynnfield, MA (US)

(21) Appl. No.: 17/603,446

(22) PCT Filed: Mar. 28, 2020
(86) PCT No.: PCT/US20/25557

§ 371 Date: Oct. 13, 2021

Related U.S. Application Data
(60) Provisional application No. 62/833,905, filed on Apr. 15, 2019.

(51) Int. Cl. C08L 39/06; C08L 21/02; A61F 6/04

(52) U.S. Cl. CPC C08L 39/06; C08L 2203/02; A61F 6/04; C08L 21/02

(57) ABSTRACT

The invention provides novel polymer formulations and use thereof in surface coatings of various products. More particularly, the invention provides a novel hydrophilic polymer and latex polymer blend coating formulation and compositions thereof, having one or more selected surfactants therein, and their use on various devices (e.g., prophylactic and medical devices) to form hydrophilic and flexible coatings with durable lubricity.

### PRIORITY CLAIMS AND RELATED PATENT APPLICATIONS

This application claims the benefit of priority from U.S. Provisional Application Ser. No. 62/833,905, filed on Apr. 15, 2019, the entire content of which is incorporated herein by reference in its entirety.

### STATEMENT REGARDING FEDERALLY FUNDED RESEARCH OR DEVELOPMENT

This invention was made with government support under Grant No. R43HD089856-01A1 awarded by the Eunice Kennedy Shiver Nation Institute of Child Health and Human Development Center of the U.S. National Institute of Health. The Government has certain rights in the invention.

### TECHNICAL FIELD OF THE INVENTION

The invention generally relates to polymer formulations and use thereof in surface coatings of various products. More particularly, the invention relates to a novel hydrophilic polymer and latex polymer blend coating formulation and compositions thereof, and their use on various devices (e.g., prophylactic and medical devices) to form hydrophilic and flexible coatings with durable lubricity.

### BACKGROUND OF THE INVENTION

Natural or synthetic latex possesses favorable physical and rheological properties due to its soft elastic characteristics and high resistance to mechanical stresses and heat. Because of these desirable properties, latex is widely incorporated in a number of consumer goods and medical devices (e.g., condoms). Due to its hydrophobicity, however, latex is not compatible with blood, and can irreversibly adsorb proteins and platelets on its surface when in contact with biologics. Additionally, high frictional and abrasive forces can occur between the interface of latex-based medical devices and tissues, leading to discomfort, increased risks of infections, tissue damage, and pain for the user.

According to the World Health Organization, the lack of adequate lubrication remains a major drawback for proper and consistent condom use. The minimal amount of lubrication included with condoms during packaging is typically insufficient to maintain the condom's lubricity throughout intercourse. Consequently, partners will either (1) not use a condom and increase risks for unwanted pregnancies and spread of Sexually transmitted infections (STIs), or (2) use an inadequately lubricious condom resulting in mucosal microtrauma, pain, and decreased satisfaction between partners. This microtrauma and pain may arise from dramatically increased penetrative force during intercourse when lubrication is inadequate. Typical healthy vaginal lubrication has been reported to require a penetrative force of 0.5 kg, whereas a typical post-menopausal or poor vaginal lubrication requires a three-fold greater penetrative force of 1.5 kg. There is also a significant demand for better lubrication products and strategies to be used with condoms, particularly for older women in menopause and post-menopausal stages affected by vaginal dryness.

Silicone-based lubricants are the most commonly used lubricants today and are applied to both male and female condoms during the manufacturing process prior to packaging. Despite this, condom consumer satisfaction with condom lubricity remains low. Unfortunately, these unmet needs exacerbate the societal stigma and negative perception that condoms are uncomfortable and will decrease pleasure during intercourse, which adversely affects the consistency of proper condom usage.

A variety of methods have been explored to chemically or physically modify natural or synthetic latex to possess hydrophilic and lubricous properties, all with limited success. The different types of hydrophilic monomers that are compatible for polymerization reactions are currently limited. Surface modification strategies involving the application of hydrophilic coatings to the surface of latex-based substrates have proven to be challenging, for example, due to persistent delamination between the coating and latex.

A surfactant can be added to the hydrophilic polymer and latex polymer blend coating formulation to improve the miscibility between the dissolved hydrophilic polymer solution and latex polymer blend solution. Surfactants are used to lower the surface tension (or interfacial tension) between two liquids given they comprise both hydrophilic and hydrophobic properties. In this case, the presence of the surfactant can help to adjust the hydrophilic polymer blend to be more compatible with the hydrophobic latex suspension in the latex polymer blend. Without the addition of the surfactant, overtime if left undisturbed, the finalized coating formulation can separate in solution, which would adversely affect the ability to apply a homogenous coating layer onto latex substrates. Continued stirring of the finalized coating formulation is practical at small scale to ensure homogeneous mixing, however at large manufacturing scale this is not practical. The presence of a surfactant was found to be useful in minimizing this separation to generate a more stabilized, homogenous coating formulation which is optimal for the coating application.

A further challenge of conventional surfactant use in manufacturing is that surfactants typically alter surface properties of liquid mixture and also of a resulting solid material formed therefrom. In many instances where only the liquid state requires the surfactant's effects and the solid state is not impacted by the presence of the surfactant, then no challenge is presented; however for the types of hydrophilic polymer coatings with durable lubricity described herein, the surface properties, namely, lubricity, of the material's solid state are of utmost importance to their use. Common surfactants and surfactant concentrations which adversely affect lubricity are therefore not useful in such an application, and the development described herein of non-obvious surfactant systems which do not adversely affect lubricity are highly useful and enable the inventive lubricious coatings to function as intended.

Thus, there is an ongoing critical need to develop novel durable, flexible, non-toxic and low-costing hydrophilic coatings and application strategies that are compatible with natural and synthetic latex-based surfaces and medical devices, particularly for condoms.

### SUMMARY OF THE INVENTION

The invention is based in part on the discovery of novel hydrophilic polymer and latex polymer blend coating formulations, and compositions thereof, having therein one or more selected surfactants. These disclosed formulations and compositions are suitable for use, for example, as hydrophilic coating on various devices, such as condoms and other prophylactic or medical devices, to form hydrophilic and flexible coatings with durable lubricity. Other devices that can benefit from such a coating include, for example, solid, non-flexible and flexible surfaces of prophylactic and biomedical latex-based devices or tools.

In one aspect, the invention generally relates to an aqueous composition, comprising: a hydrophilic polymer; a suspension of latex polymer microparticles; and a first surfactant. The hydrophilic polymer is present in the composition at a concentration from about 1 w/v % to about 10 w/v %. The latex polymer microparticles are present in the composition at a concentration from about 20 w/v % to about 60 w/v %. The surfactant is present in the composition at a concentration from about 0.01 w/v % to about 5 w/v %.

In another aspect, the invention generally relates to a cured material formed by heating or chemically curing a composition disclosed herein for a time sufficient to form interpenetrating polymer networks of hydrophilic polymer and latex polymer.

In yet another aspect, the invention generally relates to a condom produced by heating or chemically curing a composition disclosed herein for a time sufficient to form interpenetrating polymer networks of hydrophilic polymer and latex polymer.

In yet another aspect, the invention generally relates to a condom, comprising: a sheath of an elastomeric material selected from natural or synthetic rubber latex, the sheath having an outer surface and an inner surface; and a layered coating comprising a first layer disposed on and adhered to at least a portion of the outer surface of the sheath and a second layer disposed on and adhered to at least a portion of the first layer. The first layer is a cured latex polymer; and the second layer is a cured blend of a latex polymer and a hydrophilic polymer formed by heating a composition of any one of claims 1-19 for a time sufficient to form interpenetrating polymer networks of hydrophilic polymer and latex polymer.

In yet another aspect, the invention generally relates to a packaged condom product comprising one or more condoms disclosed herein. In certain embodiments, the condom and a lubricant are together. In certain embodiments, the condom and a lubricant are in separate compartments.

### BRIEF DESCRIPTION OF THE DRAWINGS

FIG. 1. Exemplary data of friction testing.

FIG. 2. Exemplary data of friction testing.

### DEFINITIONS

Unless stated otherwise, or implicit from context, the following terms and phrases include the meanings provided below. The definitions are provided to aid in describing particular embodiments, and are not intended to limit the claimed invention, because the scope of the invention is limited only by the claims. Further, unless otherwise required by context, singular terms shall include pluralities and plural terms shall include the singular. Thus, as used herein and in the claims, the singular forms include the plural reference and vice versa unless the context clearly indicates otherwise.

Other than in the operating examples, or where otherwise indicated, all numbers expressing quantities of ingredients or reaction conditions used herein should be understood as modified in all instances by the term “about.” The term “about” when used in connection with a value can mean 5% of the value being referred to. For example, about 100 means from 95 to 105.

As used herein, the term “latex” refers to natural or synthetic latex, which include vulcanized or non-vulcanized. The term “latex polymer” refers to the polymer(s) the latex is formed from. Typically, the latex substrate is hydrophobic. In some embodiments, the latex is biocompatible provided that the user does not have an adverse or allergic reaction when in contact with latex. Synthetic latex may include synthetic rubber materials, including but not limited to nitrile, hydrogenated nitrile, ethylene-propylene, fluorocarbon, chloroprene, silicone, fluorosilicone, polyacrylate, ethylene acrylic, acrylic polymers, styrenebutadiene, acrylonitrile butadiene, polyvinyl acetate, or polyurethane rubbers.

As used herein, the term “hydrophilic polymer” refers to homo- or co-polymers that exhibit hydrophilic properties, i.e., having a strong affinity for water. Non-limiting examples of hydrophilic polymers include poly(vinyl pyrrolidone)(PVP), poly(ethylene glycol) (PEG), poly(vinyl alcohol) (PVA), poly(N-i sopropylacrylamide), polyacrylamide, poly(2-oxazoline), polyethylenimine, polyacrylic acid), polymethacrylate, poly(2-ethylacrylic acid), poly(acrylic acid), poly(sulfopropyl acrylate) potassium salt, poly(2-methacryloyloxyethyl phosphorylchlorine), poly(2-propylacrylic acid), poly(methacrylic acid), poly(2-hydroxypropyl methacrylate), hydroxypropylmethylcellulose (HPMC), poly(oxanorbornene), polyelectrolytes, and co-polymers thereof.

As used herein, the term “hydrophilic latex blend” or “hydrophilic and latex polymer blend” refers to an evenly-mixed and viscous solution mixture composed of a hydrophilic polymer and latex dissolved in an aqueous solution.

As used herein, the term “non-cytotoxic” refers to biocompatibility with mammalian cells.

As used herein, the term “biocompatible” refers to the absence of an adverse acute, chronic or escalating biological response to an implant or coating, and is distinguished from a mild, transient inflammation which typically accompanies surgery or implantation of foreign objects into a living organism.

As used herein, the term “viscous” means a liquid material, e.g., a solution having viscosity of several hundreds centipoises to several millions centipoises. For example the measurement of viscosity can range from about 10² cP to about 10⁷ cP.

### DETAILED DESCRIPTION OF THE INVENTION

The invention provides a novel hydrophilic polymer and latex polymer blend coating formulation and compositions thereof having therein one or more selected surfactants. These compositions are suitable for use, for example, as hydrophilic coating on various devices, such as condoms and other prophylactic or medical devices, to form hydrophilic and flexible coatings with durable lubricity. When in contact with water or an aqueous solution, the hydrophilic coating becomes durably lubricious. This thin hydrophilic coating is able to maintain its lubricity continuously when in contact with surfaces in the presence of water without impacting on the mechanical and physical properties of the coated device. Thus, frictional forces are lowered when in contact with tissue or other interfaces. A durable and lubricious hydrophilic coating on latex condoms can lead to a reduction of condom breakage, pain, discomfort, and mucosal microtrauma for the user. Other devices that can benefit from such a coating include, for example, solid, non-flexible and flexible surfaces of prophylactic and biomedical latex-based devices or tools.

In one aspect, the invention generally relates to an aqueous composition, comprising: a hydrophilic polymer; a suspension of latex polymer microparticles; and a first surfactant. The hydrophilic polymer is present in the composition at a concentration from about 1 w/v % to about 10 w/v %. The latex polymer microparticles are present in the composition at a concentration from about 20 w/v % to about 60 w/v %. The surfactant is present in the composition at a concentration from about 0.01 w/v % to about 5 w/v %.

In certain embodiments, the weight ratio of the hydrophilic polymer to the latex polymer microparticles is in the range from about 1:30 to about 1:2.

In certain embodiments, the hydrophilic polymer has a mean molecular weight (Me) in the range from about 100 kDa to about 10,000 kDa (e.g., from about 100 kDa to about 50,000 kDa, from about 100 kDa to about 30,000 kDa, from about 100 kDa to about 10,000 kDa, from about 100 kDa to about 5,000 kDa, from about 100 kDa to about 1,000 kDa, from about 1,000 kDa to about 10,000 kDa, from about 5,000 kDa to about 10,000 kDa, from about 100 kDa to about 700 kDa, from about 300 kDa to about 700 kDa).

The hydrophilic polymer may be present in the composition at any suitable concentration in aqueous composition, for example, from about 2 w/v % to about 10 w/v % (e.g., from about 2 w/v % to about 9 w/v %, from about 2 w/v % to about 8 w/v %, from about 2 w/v % to about 7 w/v %, from about 2 w/v % to about 6 w/v %, from about 2 w/v % to about 5 w/v %, from about 3 w/v % to about 10 w/v %, from about 4 w/v % to about 10 w/v %, from about 5 w/v % to about 10 w/v %, from about 6 w/v % to about 10 w/v %, from about 3 w/v % to about 8 w/v %, from about 4 w/v % to about 9 w/v %,).

In certain embodiments, the hydrophilic polymer is present in the composition at a concentration from about 2 w/v % to about 7 w/v % (e.g., from about 2 w/v % to about 3.5 w/v %, from about 3.5 w/v % to about 5 w/v %, from about 5 w/v % to about 7 w/v %).

In certain embodiments, the surfactant is present in the composition at a concentration from about 0.01 w/v % to about 2 w/v % (e.g., 0.05 w/v % to about 2 w/v %, from about 0.1 w/v % to about 2 w/v %, from about 0.5 w/v % to about 2 w/v %, from about 1 w/v % to about 2 w/v %, from about 0.05 w/v % to about 1 w/v %, from about 0.05 w/v % to about 0.5 w/v %, about 0.05 w/v % to about 0.1 w/v %).

In certain embodiments, the composition is an evenly mixed and stable suspension.

In certain embodiments, the hydrophilic polymer comprises one or more hydrophilic polymers selected from the group consisting of: homo- or co-polymers of vinyl pyrrolidone, ethylene glycol and/or vinyl alcohol.

In certain embodiments, the hydrophilic polymer comprises one of poly(vinyl pyrrolidone)(PVP), poly(ethylene glycol) (PEG), and poly(vinyl alcohol) (PVA). In certain embodiments, the hydrophilic polymer comprises poly(vinyl pyrrolidone)(PVP). In certain embodiments, the hydrophilic polymer comprises poly(ethylene glycol) (PEG). In certain embodiments, the hydrophilic polymer comprises poly(vinyl alcohol) (PVA). In certain embodiments, the hydrophilic polymer comprises two or more of poly(vinyl pyrrolidone)(PVP), poly(ethylene glycol) (PEG), and poly(vinyl alcohol) (PVA).

In certain embodiments, the composition comprises a second hydrophilic polymer.

In certain embodiments, the composition comprises more than one surfactant, i.e., a second or additional surfactant(s).

In certain embodiments, the first surfactant, second surfactant or additional surfactant(s) if present, are selected from saturated and unsaturated fatty alcohols, for example, stearyl alcohol, oleyl alcohol, palmitoleyl alcohol, cetyl alcohol, myristyl alcohol, or lauryl alcohol.

In certain embodiments, the composition further comprises one or more of vulcanizing agents, for example, diisopropyl xanthogen polysulfide, sulfur and ammonia.

In certain embodiments, the composition further comprises one or more of accelerating agents, for example, zinc-N-diethyl-dithio-carbomate, zinc-N-dibutyl-dithio-carbomate and ammonia.

In certain embodiments, the composition further comprises one or more of antimicrobials, antivirals, antifungals, vitamins, colors, or antibiotics.

In certain embodiments, the latex polymer is natural.

In certain embodiments, the latex polymer is synthetic. In certain embodiments, the synthetic rubber latex is synthetized from isoprene, nitrile, butadiene, styrene-butadiene, chloroprene, isobutylene, or co-polymers thereof.

The latex polymer microparticles may be present in the composition at any suitable concentration in aqueous composition, for example, from about 20 w/v % to about 65 w/v % (e.g., from about 30 w/v % to about 65 w/v %, from about 40 w/v % to about 65w/v %, from about 50 w/v % to about 65 w/v %, from about 20 w/v % to about 50 w/v %, from about 20 w/v % to about 40 w/v %, from about 20 w/v % to about 30 w/v %).

In certain embodiments, the latex polymer microparticles is present in the composition at a concentration from about 35 w/v % to about 55 w/v % (e.g., from about 35 w/v % to about 40 w/v %, from about 40 w/v % to about 45 w/v %, from about 45 w/v % to about 50 w/v %, from about 50 w/v % to about 55 w/v %).

The weight ratio of the hydrophilic polymer to the latex polymer microparticles in aqueous composition may be any suitable value, for example, in the range from about 1:1 to about 1:3 (e.g., about 1:1 to about 1:2.5, about 1:1 to about 1:2, about 1:1 to about 1:1.5, about 1:1.5 to about 1:3, about 1:2 to about 1:3, about 1:2.5 to about 1:3).

The aqueous composition is preferably a well-mixed and stable suspension.

The aqueous composition is preferably a viscous aqueous composition, for example, with a viscosity in the range from about 10 cP to about 10¹⁰ cP (e.g., from about 10 cP to about 10² cP, from about 10 cP to about 10⁴ cP, from about 10 cP to about 10⁶ cP, from about 10 cP to about 10⁸ cP, from about 10² cP to about 10¹⁰ cP, from about 10⁴ cP to about 10¹⁰ cP, from about 10⁶ cP to about 10¹⁰ cP, from about 10⁸ cP to about 10¹⁰ cP, from about 10² cP to about 10⁶ cP, from about 10⁴ cP to about 10⁸ cP).

In another aspect, the invention generally relates to a cured material formed by heating or chemically curing a composition disclosed herein for a time sufficient to form interpenetrating polymer networks of hydrophilic polymer and latex polymer.

In certain embodiments, the material is a coating forming a surface of a product, for example, a condom, a sex toy, a medical device, a medical implant, apparel, or a glove. In certain embodiments, the product is a condom.

In yet another aspect, the invention generally relates to a condom produced by heating or chemically curing a composition disclosed herein for a time sufficient to form interpenetrating polymer networks of hydrophilic polymer and latex polymer.

In yet another aspect, the invention generally relates to a condom, comprising: a sheath of an elastomeric material selected from natural or synthetic rubber latex, the sheath having an outer surface and an inner surface; and a layered coating comprising a first layer disposed on and adhered to at least a portion of the outer surface of the sheath and a second layer disposed on and adhered to at least a portion of the first layer. The first layer is a cured latex polymer; and the second layer is a cured blend of a latex polymer and a hydrophilic polymer formed by heating a composition of any one of claims 1-19 for a time sufficient to form interpenetrating polymer networks of hydrophilic polymer and latex polymer.

In certain embodiments, the condom further comprises a silicon-based or water-based lubricant applied to the second layer.

In certain embodiments, the condom further comprises a coating of powders or dusting agents, selected from cornstarch, baby powder, or talc, applied to the second layer.

In certain embodiments, upon contacting with moisture or water, the second layer becomes slippery with durable lubricity.

In yet another aspect, the invention generally relates to a packaged condom product comprising one or more condoms disclosed herein. In certain embodiments, the condom and a lubricant are together. In certain embodiments, the condom and a lubricant are in separate compartments.

It is noted that the surfactant can be an anionic, neutral, zwitterionic or cationic reagent. In a more preferred embodiment, the surfactant can be neutral. In the most preferred embodiment, the surfactant is compatible and dissolved in alkaline conditions (pH>9) to be compatible with the latex blend composition. In another embodiment, the surfactant can be a polymer, lipid, bio reagent, Sulfobetaine, amphiphiles, or long chain amino acids.

In one embodiment, the surfactant can be but is not limited to polyethylene glycol mono-isotridecyl ether sulfate, sodium salt, 4-[2-[2-(2-Alkoxy(C12-C15) ethoxy) ethoxy]ethyl]disodium sulfosuccinate, SDS, Lauryldimethylamine oxide, Stearamine Oxide (5% limit) , Polysorbate 20 (non-ionic surfactant), Tween 20, Triton X -100, Oleyl alcohol, Nonoxyl-9, spermicide, lipids, and phospholipids, lauric acid, zinc dialkyl dithiocarbamate, straight-chain long alkyl groups (C8-C20), branched-chain long alkyl groups (C8-H20), long-chain alkyl benzene residues (C8-H15), alkyl napthalene residues (C3 and greater length alkyl groups), rosin derivatives, high molecular weight propylene oxide polymers (polyoxypropylene glycol derivatives), long chain perfluoroalkyl groups, lipids comprise of sterols and sterol esters (approx.0.4%), wax and phospholipids, and those with polysiloxane groups.

In a more preferred embodiment, the surfactant may consist of but is not limited to SDS, Tween 20, or olyel alcohols.

In a certain embodiment the surfactant can be added and mixed with the hydrophilic polymer blend prior towards adding it with the latex suspension to generate the final hydrophilic polymer and latex polymer blend coating formulation. In another embodiment the surfactant can be added and mixed to the latex suspension prior to mixing with the hydrophilic polymer blend. In another embodiment the addition of the surfactant can done after preparing the finalized hydrophilic polymer and latex polymer blend coating formulation and mixed well to generate a homogenous solution. In one embodiment, the surfactant is compatible to the latex and hydrophilic reagents.

In a certain embodiment, the surfactant should be added at a concentration less than than 5 v/v % of the final coating formulation. In another embodiment, the surfactant should be added is less than 1 w/v %. In a more preferred embodiment. In a more preferred embodiment the surfactant should be added is less than 1 w/v %.

In case the surfactant is a solid, this should dissolve in an aqueous solution which can then be added to the hydrophilic polymer blend prior towards adding it with the latex suspension.

In one embodiment the addition of the surfactant can form a homogenous solution or an emulsified solution. The addition of the surfactant should not create precipitants or solids in solution.

The compositions disclosed herein may further include one or more surfactants.

In one embodiment, the surfactant may affect the viscosity of the final coating blend formulation.

In one embodiment, the surfactant may increase the hydrophilicity or wetting properties of the final coating blend formulation.

In a certain embodiment, the addition of the surfactant must not significant affect the resulting lubricity of the polymeric coating after application onto a latex substrate. The polymeric coating should have similar lubricating properties and durability comparable to a coating blend formulation that does not comprise of the surfactant.

In a preferred embodiment, the addition of the surfactant does not change the mechanical properties, flexibility, or strength of the finalized coated substrate after heat curing. In a most preferred embodiment, the finalized coating should have similar mechanical properties and flexibility that is comparable to preparing the formulation with or without the surfactant.

### EXAMPLES

### Surfactant Screening and Identification

For this initial screening, different surfactants were investigated to evaluate their compatibility with the hydrophilic polymer and latex polymer blend coating formulation. For an optimal formulation, the presence of a surfactant should result in a homogenous, smooth, well-mixed solution without forming precipitate (curing of latex particles) or the hydrophilic polymer crashing out of solution. The resulting formulation should still be applicable onto latex substrates in a similar manner as to formulations without the surfactant. Importantly, the addition of the surfactant should not reduce the lubricity properties, durability of the coating, or material integrity after being applied and cured on the latex

| | | (2) | | | |
|---|---|---|---|---|---|
| | (1) | Emulsion | | | |
| | Homogeneity | formation in | | | (5) |
| | of the final | hydrophilic | (3) Changes | (4) Separation | Precipitate |
| | solution | polymer blend | in viscosity | after 12 hours | formation |
| No surfactant | Yes (Pass) | No | NA (control) | Yes (Fail) | No (Pass) |
| SDS in water | Yes (Pass) | No | No | Slightly | No (Pass) |
| | | | (Desirable) | (Intermediate) | |
| Polysorbate | Yes (Pass) | Slightly | Yes | Slightly | No (Pass) |
| 20/Tween 20 | | | (Undesirable) | (Intermediate) | |
| Triton X-100 | Yes (Pass) | Yes | Yes | No (Pass) | Yes (Fail) |
| | | | (Undesirable) | | |
| Oleyl Alcohol | Yes (Pass) | Yes | Slightly | No (Pass) | No (Pass) |
| | | | (Intermediate) | | |

In this study, the qualitative factors investigated were: (1) homogeneity of the final solution; (2) formation of an emulsion when mixed with the hydrophilic polymer blend solution; (3) changes in viscosity of the final hydrophilic polymer and latex polymer blend coating formulation compared to samples without the surfactant; (4) observable separation between the hydrophilic polymer blend and latex polymer blend after 12 hours; and, (5) presence of precipitates in the final formulation. All samples were prepared with a mixture of a 1:1 volumetric ratio of the hydrophilic polymer formulation and the latex polymer blend, total volume 15 mL). Prior to mixing the two components to produce the final formulation, 10 drops of each surfactant (in excess) using a 1 mL Luer slip syringe were added and mixed in the hydrophilic polymer blend first. All samples were compared to a control sample that was prepared in the same manner without a surfactant. Observations from the initial screening are summarized in the table below.

Results from this initial screening indicated compatible surfactants can be SDS, Polysorbate 20, or oleyl alcohol.

### Optimization of the Hydrophilic Polymer and Latex Polymer Blend Coating Formulation in Presence of a Surfactant

Based on results from the above experimental study, three surfactants were further investigated to determine its compatibility with the hydrophilic polymer and latex polymer blend coating formulation. In this following screening, all samples were prepared in a similar manner as above. The amount of surfactant added varied among 10, 20, or 30 drops, which was added using a 1 mL Luer slip syringe. All samples were compared to control samples of the coating formulation without surfactant. Similar qualitative observations were noted as above and recorded in the table as followed.

| | | | (2) | | | |
|---|---|---|---|---|---|---|
| | | (1) | Emulsion | (3) | (4) | |
| | | Homogeneity | formation in | Changes | Separation | (5) |
| | Drops of | of the final | hydrophilic | in | after 12 | Precipitate |
| | surfactant | solution | polymer blend | viscosity | hours | formation |
| 1M SDS in | 10 | Yes | No | No | Slightly | No |
| water | | | | | (Intermediate) | |
| | 20 | Yes | No | No | Slightly | No |
| | | | | | (Intermediate) | |
| | 30 | Yes | No | No | Slightly | No |
| | | | | | (Intermediate) | |
| Polysorbate | 10 | Yes | Slightly | Yes | Slightly | No |
| 20 | | | | | (Intermediate) | |
| | 20 | Yes | Yes | Yes | Slightly | No |
| | | | | | (Intermediate) | |
| | 30 | Yes | Yes | Yes | No (Pass) | No |
| Oleyl | 10 | Yes | Yes | Slightly | No (Pass) | No |
| Alcohol | 20 | Yes | Yes | Yes | No (Pass) | No |
| | 30 | Yes | Yes | Yes | No (Pass) | No |

Results from this follow-up screening indicated the most compatible surfactant to be oleyl alcohol.

### Optimization of the Hydrophilic Polymer and Latex Polymer Blend Coating Formulation in Presence of a Surfactant and Scale-Up

Out of the surfactants investigated in the screening, the findings indicated oleyl alcohol would be the most compatible surfactant for the hydrophilic polymer and latex polymer blend coating formulation. To prepare an optimal formulation for dip-coating applications, it is important to note that the preparation of the latex stock solution (e.g., amount of solid microparticles of latex content, the vulcanized latex preparation, freshness of the latex batch) will influence the properties of the final formulation in presence of the surfactant. Thus the final concentration range of the surfactant may vary between batch-to-batch samples.

A follow-up experiment was conducted to evaluate the effects of adding oleyl alcohol to the formulation in a slightly larger volume (50 mL). All samples were prepared in a similar manner as above. The amount of oleyl alcohol varied and was added using a syringe. All samples were compared to control samples of the coating formulation without surfactant. Similar qualitative observations were noted as above and recorded in the table as followed.

| | Oleyl alcohol | | | | | |
|---|---|---|---|---|---|---|
| | concentration | (1) | | | (4) | |
| Volume of | (v/v) in final | Homogeneity of | (2) | (3) | Separation | (5) |
| Oleyl | coating | the final | Emulsion | Changes in | after 12 | Precipitate |
| alcohol | formulation | solution | formation | viscosity | hours | formation |
| 0 | 0% | Yes | Yes | No | Yes | No |
| 0.375 mL | 1.25% | Yes | Yes | Slightly | Slightly | No |
| 0.75 mL | 2.5% | Yes | Yes | Slightly | No | No |
| 1.5 mL | 5% | Yes | Yes | Yes (very | No | No |
| | | | | viscous) | | |

Results from this optimization study indicated that a final oleyl alcohol concentration of 2.5 v/v % was superior to 1.25 v/v % (not enough surfactant to fully prevent separation from occurring) and to 5 v/v % (sufficient surfactant concentration to increase viscosity adversely), and that a concentration between 1.25 and 5 v/v % may be optimal.

Friction evaluation of hydrophilic polymer and latex polymer blend formulation with or without surfactant applied on latex condoms

It is important to confirm the addition of the surfactant in the formulation does not hinder the lubricating properties of the resulting coating after its application onto latex substrates and when in contact with water. For this investigation, latex condom prototypes were prepared in a similar manner as previously described. The formulation with oleyl alcohol (0.01 v/v %) was prepared on a 500 mL scale for these assessments. A 18 mm diameter glass mandrel was placed into a latex solution to form the first base coat layer via dip-coating which was cured via heat and then followed by the second hydrophilic and latex polymer blend layer dip to generate a homogenous and thin coating. After the coating was cured via heat, the outer surface was lightly dusted with cornstarch to prevent sticking. Control samples were also prepared in a similar manner that was not coated with the hydrophilic polymer and latex polymer blend coating formulation, as well as samples coated with the hydrophilic polymer and latex polymer blend coating formulation that does not contain the surfactant.

### Friction Testing Method

A custom-built friction-measuring device (“Reciprocator”) was used to determine condom lubricity by measuring the frictional force between a condom (on a cylindrical glass mandrel) and a tissue-like countersurface (lining the interior surface of a cylindrical bearing housing) upon repeated insertion and withdrawal (i.e. reciprocation). The Reciprocator contains a reciprocating linear actuator to move the glass mandrel forward and reverse, and a cylindrical housing attached to an axial force gauge to measure the force of insertion and withdrawal of the mandrel into and out of the cylindrical housing. Force data is transmitted to computer for analysis.

To perform testing, first the condom was unrolled onto the glass mandrel and the mandrel was submerged in water nearly to the top of the condom's beaded retaining ring for ten seconds to wet the condom. Then the mandrel was removed from water and excess water was allowed to drip off (five seconds). Water was applied to the housing interior to fully wet it; excess water was allowed to drain away from the housing. Data collection was programmed via computer to record frictional force at a collection frequency of 20 Hz. The mandrel was positioned at the opening of the housing and the actuator was driven forward approximately one inch to seat the tip of the mandrel within the housing. Reciprocations were performed by switching the actuator between forward and reverse directions at 1 Hz (reciprocation frequency 0.5 Hz). Stroke length was 5.5″. Upon completion of the test (200 forward-reverse cycles), data was exported to tabular force-versus-time format for analysis. The local maximum absolute force values for the forward direction (reverse direction values did not differ significantly) are plotted versus time.

### Friction Testing Results

The samples prepared as described and that were tested for lubricity were as follows.

| | Final formulation PVP | Final formulation Oleyl |
|---|---|---|
| Sample name | concentration | alcohol concentration |
| Commercial non-lubricated | NA (0%) | NA (0%) (control) |
| condom (Trojan ENZ) | | |
| “HGC 1x (ctrl)” | 2.5 w/v % | 0 v/v % (control) |
| “HGC 1x, O.A. 1.25%” | 2.5 w/v % | 1.25 v/v % |
| “HGC 1x, O.A. 2.5%” | 2.5 w/v % | 2.5 v/v % |
| “HGC 2x (ctrl)” | 5 w/v % | 0 v/v % (control) |
| “HGC 2x, O.A. 1.25%” | 5 w/v % | 1.25 v/v % |
| “HGC 2x, O.A. 2.5%” | 5 w/v % | 2.5 v/v % |

Comparing among the “HGC 1×” samples, presence of 1.25% O.A. did not worsen friction compared to control. The presence of 2.5% O.A. worsened friction by a small but not significant amount.

Comparing among the “HGC 2×” samples, there was a similar finding there was no significant effect of the surfactant on friction.

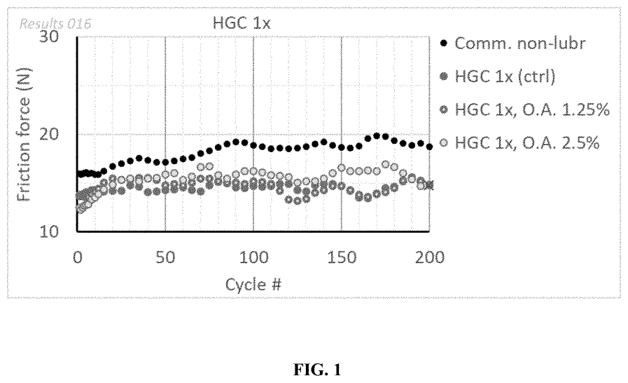

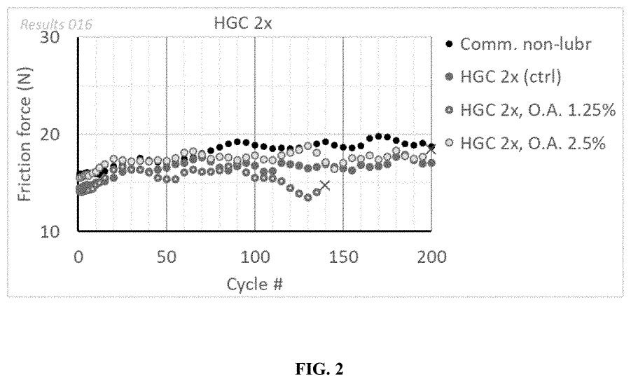

Exemplary results are presented in FIG. 1 and FIG. 2.

### CONCLUSIONS

There are no significant changes in friction when O.A. is present. 1.25% O.A. shows negligible difference from control, while 2.5% shows a small yet not significant increase in friction.

Applicant's disclosure is described herein in preferred embodiments with reference to the Figures, in which like numbers represent the same or similar elements. Reference throughout this specification to “one embodiment,” “an embodiment,” or similar language means that a particular feature, structure, or characteristic described in connection with the embodiment is included in at least one embodiment of the present invention. Thus, appearances of the phrases “in one embodiment,” “in an embodiment,” and similar language throughout this specification may, but do not necessarily, all refer to the same embodiment.

The described features, structures, or characteristics of Applicant's disclosure may be combined in any suitable manner in one or more embodiments. In the description, herein, numerous specific details are recited to provide a thorough understanding of embodiments of the invention. One skilled in the relevant art will recognize, however, that Applicant's composition and/or method may be practiced without one or more of the specific details, or with other methods, components, materials, and so forth. In other instances, well-known structures, materials, or operations are not shown or described in detail to avoid obscuring aspects of the disclosure.

In this specification and the appended claims, the singular forms “a,” “an,” and “the” include plural reference, unless the context clearly dictates otherwise. Ranges provided herein are understood to be shorthand for all of the values within the range. For example, a range of 1 to 50 is understood to include any number, combination of numbers, or sub-range from the group consisting 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, or 50. As used herein, “at least” a specific value is understood to be that value and all values greater than that value. As used herein, “more than one” is understood as 2, 3, 4, 5, 6, 7, 8, 9, 10, 11, 12, 13, 14, 15, 16, 17, 18, 19, 20, 25, 30, 40, 50, 100, etc., or any value therebetween. Unless specifically stated or obvious from context, as used herein, the term “or” is understood to be inclusive.

Unless defined otherwise, all technical and scientific terms used herein have the same meaning as commonly understood by one of ordinary skill in the art. Although any methods and materials similar or equivalent to those described herein can also be used in the practice or testing of the present disclosure, the preferred methods and materials are now described. Methods recited herein may be carried out in any order that is logically possible, in addition to a particular order disclosed.

### INCORPORATION BY REFERENCE

References and citations to other documents, such as patents, patent applications, patent publications, journals, books, papers, web contents, have been made in this disclosure. All such documents are hereby incorporated herein by reference in their entirety for all purposes. Any material, or portion thereof, that is said to be incorporated by reference herein, but which conflicts with existing definitions, statements, or other disclosure material explicitly set forth herein is only incorporated to the extent that no conflict arises between that incorporated material and the present disclosure material. In the event of a conflict, the conflict is to be resolved in favor of the present disclosure as the preferred disclosure.

### EQUIVALENTS

The representative examples are intended to help illustrate the invention, and are not intended to, nor should they be construed to, limit the scope of the invention. Indeed, various modifications of the invention and many further embodiments thereof, in addition to those shown and described herein, will become apparent to those skilled in the art from the full contents of this document, including the examples and the references to the scientific and patent literature included herein. The examples contain important additional information, exemplification and guidance that can be adapted to the practice of this invention in its various embodiments and equivalents thereof.

1. An aqueous composition, comprising:
a hydrophilic polymer;
a suspension of latex polymer microparticles; and
a first surfactant; wherein
the hydrophilic polymer is present in the composition at a concentration from about 1 w/v % to about 10 w/v %;
the latex polymer microparticles are present in the composition at a concentration from about 20 w/v % to about 60 w/v %; and
the surfactant is present in the composition at a concentration from about 0.01 w/v % to about 5 w/v %.

2. The composition of claim 1, wherein the weight ratio of the hydrophilic polymer to the latex polymer microparticles is in the range from about 1:30 to about 1:2.

3. The composition of claim 1, wherein the hydrophilic polymer has a mean molecular weight in the range from about 100 kDa to about 700 kDa.

4. The composition of claim 1, wherein the surfactant is present in the composition at a concentration from about 0.1 w/v % to about 2 w/v %.

5. The composition of claim 1, being an evenly mixed and stable suspension.

6. The composition of claim 1, wherein the hydrophilic polymer comprises one or more hydrophilic polymers selected from the group consisting of: homo- or co-polymers of vinyl pyrrolidone, ethylene glycol and/or vinyl alcohol.

7. The composition of claim 1, wherein the hydrophilic polymer comprises one of poly(vinyl pyrrolidone)(PVP), poly(ethylene glycol) (PEG), and poly(vinyl alcohol) (PVA).

8. The composition of claim 1, wherein the composition comprises a second hydrophilic polymer.

9. The composition of claim 1, wherein the composition comprises a second surfactant.

10. The composition of claim 1, wherein the first surfactant, or second surfactant if present, is selected from saturated and unsaturated fatty alcohols.

11. The composition of claim 10, wherein the fatty alcohols are selected from stearyl alcohol, oleyl alcohol, palmitoleyl alcohol, cetyl alcohol, myristyl alcohol, and lauryl alcohol.

12. The composition of claim 1, further comprising one or more of vulcanizing agents.

13. The composition of claim 12, wherein the vulcanizing agents are selected from the group consisting of: diisopropyl xanthogen polysulfide, sulfur and ammonia.

14. The composition of claim 1, further comprising one or more of accelerating agents.

15. The composition of claim 14, wherein the accelerating agents are selected from the group consisting of: zinc-N-diethyl-dithio-carbomate, zinc-N-dibutyl-dithio-carbomate and ammonia.

16. The composition of claim 1, further comprising one or more of antimicrobials, antivirals, antifungals, vitamins, colors, or antibiotics.

17-18. (canceled)

19. The composition of claim 18, wherein the synthetic rubber latex is synthetized from isoprene, nitrile, butadiene, styrene-butadiene, chloroprene, isobutylene, or co-polymers thereof.

20. A cured material formed by heating or chemically curing a composition of claim 1 for a time sufficient to form interpenetrating polymer networks of hydrophilic polymer and latex polymer.

21-23. (canceled)

24. A condom produced by heating or chemically curing a composition of claim 1 for a time sufficient to form interpenetrating polymer networks of hydrophilic polymer and latex polymer.

25-28. (canceled)

29. A packaged condom product comprising one or more condoms of claim 24.

30-31. (canceled)
